(19) United States
(12) Patent Application Publication
(10) Pub. No.: US 2010/0304987 A1
(43) Pub. Date: Dec. 2, 2010

(54) METHODS AND KITS FOR DIAGNOSIS AND/OR PROGNOSIS OF THE TOLERANT STATE IN LIVER TRANSPLANTATION

(76) Inventors: Alberto Sanchez Fueyo, Barcelona (ES); Marc Martinez Llordella, Barcelona (ES); Isabel Puig Pey, Barcelona (ES); Antoni Rimola Castella, Barcelona (ES); Juan Jose Lozano Salvatella, Barcelona (ES)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 12/522,120

(22) PCT Filed: Jan. 4, 2008
(86) PCT No.: PCT/EP2008/050057

§ 371 Date: Jan. 15, 2010

(30) Foreign Application Priority Data
Jan. 4, 2007 (ES) P 200700073

(51) Int. Cl. C40B 30/04; C12Q 1/68; C40B 40/06

(52) U.S. Cl. 506/9; 435/6; 506/16

(57) ABSTRACT

The long-term survival of transplanted grafts critically depends on the life-long administration of immunosuppressive drugs to prevent graft rejection. These drugs are very effective at preventing graft rejection, but they are also associated with severe side effects. Inventors have selected a set of genes whose expression characterizes the tolerant state in liver transplantation in humans. Based on the expression level profile of this set of genes, inventors provide a non-invasive method to assess diagnosis and/or prognosis of the tolerant state in liver transplantation in humans, and kits to perform it. These kits are simpler and cheaper than others based on a great number of genes, such as commercial microarrays with thousands of probes.

This invention refers to the field of human medicine, and specifically to the 5 diagnosis and/or prognosis of the tolerant state in a particular transplantation.

### BACKGROUND ART

The long-term survival of transplanted grafts critically depends on the life-long administration of immunosuppressive drugs to prevent graft rejection. These drugs are very effective at preventing graft rejection, but they are also associated with severe side effects, such as nephrotoxicity, an augmented risk of opportunistic infections and tumors, and metabolic complications such as diabetes, hyperlipidemia and arterial hypertension. Due to the side effects of immunosuppressive drugs, the induction of tolerance, defined as a state in which the graft maintains a normal function in the absence of chronic immunosuppression, is one of the main goals of research in transplant immunology. Tolerance induction is possible in a great number of experimental models of transplant in rodents. Nevertheless, the application of these experimental treatments in the clinic has been a failure to a large extent. Liver transplantation is the only clinical setting in which tolerance spontaneously occurs in a substantial proportion of patients. Indeed, complete immunosuppression withdrawal can be achieved in around 21% of patients (cf. J. Lerut et al., “An appraisal of tolerance in liver transplantation”, American Journal of Transplantation 2006, vol. 6, pp. 1774-80). Unfortunately, there are currently no means to identify these patients before immunosuppression withdrawal is attempted. For this reason, complete discontinuation of immunosuppressive drugs is rarely attempted in liver transplantation, and thus many patients continue to be unnecessarily immunosuppressed, with the health and economic problems that this involves.

One of the reasons why clinical application in humans of experimental treatments of tolerance induction has not been successful relates to the lack of an accurate tool to non-invasively diagnose tolerance in human transplant recipients. Recent publications point out the urgent need for this tool (e.g. N. Najafian et al., “How can we measure immunologic tolerance in humans?”, J. Am. Soc. Nephrol. 2006, vol. 17, pp. 2652-63; K. A. Newell et al., “Tolerante assays: measuring the unknown”, Transplantation 2006, vol. 81, pp. 1503-9).

Prior attempts to identify tolerance in transplantation, mainly of kidney and liver, have employed either antigen-specific functional assays or antigen-nonspecific tests. In the functional assays recipient T lymphocytes are challenged with donor antigens either in vitro or in vivo (cf. J. Cai et al., “Minor H antigen HA-1-specific regulator and effector CD8+T cells, and HA-1 microchimerism, in allograft tolerance”, J. Exp. Med. 2004, vol. 199, pp. 1017 23; E. Jankowska-Gan E et al., “Human liver allograft acceptance and the “tolerance assay”, Hum. Immunol. 2002, vol. 63, pp. 862-70; M. Hernández-Fuentes et al., “Indices of tolerance: Interim report”, abstract #999, World Transplant Congress 2006). These assays are very valuable from a mechanistic point of view, since they are the only tests capable of revealing which pathways are responsible for the specificity of the tolerance state. Unfortunately, these assays are also difficult to perform, highly variable from laboratory to laboratory (difficult to standardize), and require the availability of carefully cryopreserved donor cells. For these reasons, functional assays are not optimal for widespread clinical application, and are currently employed only in selected, highly specialized laboratories, and basically for research purposes.

The antigen-nonspecific immune monitoring tests constitute a variety of methodologies aiming at the phenotypic characterization of the recipient immune system, without the use of donor antigen challenges. Among these tests, the study of T cell receptor CDR3 length distribution patterns (TcLand) and peripheral blood cell immunophenotyping employing flow cytometry, have been employed to identify biomarkers characteristic of tolerance in humans. The TcLand technique has been employed in peripheral blood to discriminate between tolerant kidney recipients and recipients experiencing chronic rejection (cf. S. Brouard et al., “Operationally tolerant and minimally immunosuppressed kidney recipients display strongly altered blood T-cell clonal regulation”, Am. J. Transplant. 2005, vol. 5, pp. 330-40). However, this technique is expensive, is currently only available at one laboratory (Inserm 643 and TcLand Biotech in Nantes, France), and has never been validated in liver transplantation. The use of peripheral blood immunophenotyping has been used with peripheral blood samples from both liver and kidney tolerant transplant recipients. Two studies addressing this methodology are known to inventors. In the first one, from the University of Pittsburgh in USA (cf. G. V. Mazariegos et al., “Dendritic cell subset ratio in peripheral blood correlates with successful withdrawal of immunosuppression in liver transplant patients”, Am. J. Transplant. 2003, vol. 3, pp. 689-96), it is said that the ratio between pDC and mDC dendritic cell subsets could discriminate between tolerant and non-tolerant recipients in pediatric liver transplantation. In the second study, from Kyoto (cf. Y. Li et al., “Analyses of peripheral blood mononuclear cells in operational tolerance after pediatric living donor liver transplantation”, Am. J. Transplant. 2004, vol. 4, pp. 2118-25), it is said that an increased ratio between delta-1 and delta-2 gammadelta T cells in peripheral blood is more prevalent in tolerant than in non-tolerant liver recipients. However, none of these tests offers the accuracy required for the widespread clinical application.

While the chronic use of immunosuppressive drugs is currently the only means to ensure long-term survival of transplanted allografts, these drugs are expensive and are associated with severe side effects (nephrotoxicity, tumor and infection development, diabetes, cardiovascular complications, etc.) that lead to substantial morbidity and mortality. Hence, any strategy capable of significantly reducing the use of immunosuppressive drugs in transplantation may have a large impact on the health and quality of life of transplant recipients.

### SUMMARY OF THE INVENTION

Inventors have selected a set of genes, the expression of which characterizes the tolerant state in liver transplantation in humans. Based on the expression level profile of this set of genes, inventors provide a non-invasive method to assess diagnosis and/or prognosis of the tolerant state in liver transplantation in humans.

In this context, it is relevant to mention that gene expression profiling has been employed in both kidney and heart transplantation to identify rejection, and furthermore it has been proposed to detect tolerance in kidney transplantation (cf. S. Brouard, et al., “Identification of a peripheral blood transcriptional biomarker panel associated with operational renal allograft tolerance”, Proc. Natl. Acad. Sci. 2007, vol. 39, pp. 15448-53). Specifically in liver transplantation, one study about gene expression profiles of lymphocytes obtained from tolerant rat liver recipients has been reported (cf. Fujino et al., “Differences in lymphocyte gene expression between tolerant and syngeneic liver grafted rats”, Liver Transpl. 2004, vol. 10, pp. 379-91). However, gene expression technologies to detect tolerance have not been previously employed in human liver transplantation.

The invention provides a method of assessing diagnosis and/or prognosis of the tolerant state in liver transplantation in a human patient, comprising the steps of: (a) obtaining a biological sample from the patient; and (b) measuring the expression levels in the sample of a set of genes comprising the following twenty two: transforming growth factor beta receptor III (TGFBR3, NCBI Gene ID 7049), killer cell lectin-like receptor subfamily B member 1 (KLRB1, NCBI Gene ID 3820), asparagine-linked glycosylation 8 homolog (ALG8, NCBI Gene ID 79053), Fanconi anemia complementation group G (FANCG, NCBI Gene ID 2189), gem associated protein 7 (GEMIN7, NCBI Gene ID 79760), natural killer cell group 7 sequence (NKG7, NCBI Gene ID 4818), RAD23 homolog B of Saccharomyces cerevisiae (RAD23B, NCBI Gene ID 5887), SLAM family member 7 (SLAMF7, NCBI Gene ID 57823), TP53 regulated inhibitor of apoptosis 1 (TRIAP1, NCBI Gene ID 51499), protein phosphatase 1B magnesium-dependent beta isoform (PPM1B, NCBI Gene ID 5495), chromosome 10 open reading frame 119 (C10orf119, NCBI Gene ID 79892), T cell receptor delta locus (TRD@, NCBI Gene ID 6964), nucleolar protein family A member 1 (NOLA1, NCBI Gene ID 54433), DCN1 defective in cullin neddylation 1 domain containing 1 of Saccharomyces cerevisiae (DCUN1D1, NCBI Gene ID 54165), dystrobrevin binding protein 1 (DTNBP1, NCBI Gene ID 84062), N-acetylglucosamine-1-phosphate transferase alpha and beta subunits (GNPTAB, NCBI Gene ID 79158), proteasome 26S subunit non-ATPase 14 (PSMD14, NCBI Gene ID 10213), coatomer protein complex subunit zeta 1 (COPZ1, NCBI Gene ID 22818), S100 calcium binding protein A10 (S100A10, NCBI Gene ID 6281), ataxin 10 (ATXN10, NCBI Gene ID 25814), G-rich RNA sequence binding factor 1 (GRSF1, NCBI Gene ID 2926), and CD244 molecule natural killer cell receptor 2B4 (CD244, NCBI Gene ID 51744); wherein the corresponding gene expression levels above or below pre-determined cut-off levels are indicative of the tolerant state in liver transplantation.

As an embodiment of the invention, the expression of a second set of genes can be additionally measured. This second set of genes comprises the genes shown in Table 2. For that additional set of genes the following screening steps have been carried out: 1) analysis of additional blood samples from tolerant and non-tolerant liver transplant recipients; 2) use of a supplementary computational strategy based on the “Misclassified Posterior Probablility” (MiPP) algorithm in order to allow the prediction of tolerance based on a minimum number of genes; and 3) confirmation of all gene expression measurements employing real time reverse transcription PCR. In one preferred embodiment of the invention a subset of genes selected for their highest predictability either taken individually or in combination thereof, comprises the genes shown in Table 3.

References to genes are made with the name, symbol and Gene ID of the NCBI.

As used herein, the term “tolerant state” means the acceptance of a transplanted liver maintaining normal function in the absence of on-going immunosuppressive therapy. For the purposes of the current invention the terms “tolerance” and “operational tolerance” are considered as equivalent. The term “diagnosis” refers to the identification of tolerant patients among liver recipients receiving maintenance immunosuppressive therapy. The term “prognosis” means the capacity to predict the successful reduction/discontinuation of immunosuppressive therapy in liver recipients before treatment modification is attempted.

In an embodiment of the present invention, the gene expression levels are above pre-determined cut-off levels obtained from a control sample. In a particular embodiment, the control sample is obtained from a non-tolerant liver transplant recipient requiring on-going immunosuppression therapy, that can be called immunosuppression-dependent or non-tolerant (Non-TOL).

The differentially expressed genes are either up-regulated or down-regulated in a defined state. “Up-regulation” and “down-regulation” are relative terms meaning that a detectable difference (beyond the contribution of noise in the system used to measure it) is found in the amount of expression of the genes relative to some baseline. In this case, the baseline is the measured gene expression of the control sample. The genes of interest in the tolerant state are up regulated relative to the baseline level using the same measurement method.

The present invention provides means to use quantitative gene expression to diagnose tolerant liver transplant recipients before immunosuppressive drug withdrawal or reduction is attempted. The main application of this is the diagnosis of tolerant liver transplant recipients among patients receiving chronic immunosuppressive therapy. Consequently, it permits the dose reduction or discontinuation of immunosuppressive drugs in those patients identified as tolerant without undergoing rejection. This can result in a substantial decrease in the morbidity/mortality of drug-related side effects. This also means a significant decrease in the financial costs of therapy after liver transplantation.

Measuring the expression levels of the genes in the sample can be carried out over the transcripts of these genes (messenger RNA) or over the translation products, i.e. the proteins.

In a particular embodiment, measuring the gene expression levels is carried out using a microarray or a gene chip which comprises nucleic acid probes. Said nucleic acid probes comprise sequences that specifically hybridize to the transcripts of the set of genes defined above. At least one probe for each of the transcript must be on the microarray or the gene chip for detecting all the genes defined above, but it is possible to have more than one probe for the same transcript.

The term “specifically hybridize to” refers to the binding, duplexing, or hybridizing of a molecule substantially to or only to a particular nucleotide sequence or sequences under stringent conditions when that sequence is present in a complex mixture (e.g., total cellular DNA or RNA). “Hybridization” refers to the process in which two single-stranded polynucleotides bind non-covalently to form a stable double-stranded polynucleotide.

Microarray technology measures mRNA levels of many genes simultaneously thereby presenting a powerful tool for identifying gene expression profiles for a disease or a specific state. Two microarray technologies are currently in wide use. The first are complementary DNA (cDNA) microarrays and the second are oligonucleotide microarrays. Although differences exist in the construction of these chips, essentially all downstream data analysis and output are the same. Typically, a nucleic acid sample is prepared from appropriate source and labeled with a signal moiety, such as a fluorescent label. The sample is hybridized with the microarray under appropriate conditions. The microarrays are then washed or otherwise processed to remove non-hybridized sample nucleic acids. The hybridization is then evaluated by detecting the distribution of the label on the chip. The distribution of label may be detected by scanning the microarrays to determine fluorescence intensity distribution. Typically, the hybridization of each probe is reflected by corresponding pixel intensities. The signal intensity is proportional to the cDNA amount, and thus mRNA, expressed in the sample. Analysis of the differential expression levels is conducted by comparing such intensities for the test sample and for the control sample. A ratio of these intensities indicates the fold-change in gene expression between the test and control samples.

In a particular embodiment of the invention, the microarray is a cDNA microarray. In this format, probes of cDNA (˜500-5000 bases long) are immobilized to a solid surface, e.g., glass, using robot spotting and exposed to a set of targets either separately or in a mixture. This method, traditionally called DNA microarray, was developed at Stanford University.

In another particular embodiment, the microarray is an oligonucleotide microarray. In this format, oligonucleotides (˜20-80-mer) or peptide nucleic acid (PNA) probes are synthesized either in situ (on-chip) or by conventional synthesis followed by on-chip immobilization. The microarray is exposed to labeled sample DNA, hybridized, and the identity/abundance of complementary sequences are determined. This method, historically called DNA chip, was developed by Affymetrix, Inc., which sells its photolithographically fabricated products under the GeneChip® trademark. Many companies are manufacturing oligonucleotide based chips using alternative in-situ synthesis or depositioning technologies.

The microarray can assume a variety of formats, e.g., libraries of soluble molecules; and libraries of compounds tethered to resin beads, silica chips, on glass or other solid supports. A number of different microarray configurations, supports and production methods are known to those skilled in the art. Probes may be prepared by any method known in the art, including synthetically or grown in a biological host. Synthetic methods include but are not limited to oligonucleotide synthesis, riboprobes, and polymerase chain reaction (PCR). The probes may be labeled with a detectable marker by any method known in the art. Methods for labeling probes include random priming, end labeling, PCR and nick translation.

In a particular embodiment, the microarray or the gene chip further comprises one or more internal control probes that act for example, as normalization control probes, expression level control probes and mismatch control probes. Normalization controls provide a control for variations in hybridization conditions, label intensity, “reading” efficiency and other factors that may cause the signal of a perfect hybridization to vary between microarrays. Expression level controls are probes that hybridize specifically with constitutively expressed genes in the analyzed sample (“housekeeping genes”). Mismatch controls are oligonucleotide probes identical to their corresponding test or control probes except for the presence of one or more mismatched bases. Mismatch probes thus provide a control for non-specific binding or cross hybridization to a nucleic acid in the sample other than the target to which the probe is directed (false positives).

In other embodiments of the invention, measuring the gene expression levels of the genes is carried out by reverse transcription PCR (RT-PCR), competitive RT-PCR, real time RT-PCR, differential display RT-PCR, Northern Blot analysis and other related tests. In a particular embodiment of the invention, measuring the gene expression levels is carried out by quantitative reverse transcription PCR of RNA extracted from the sample. In a more particular embodiment, the RT-PCR comprises one or more internal control reagents. Another option is to conduct these techniques of gene expression quantification using PCR reactions, to amplify cDNA or cRNA produced from mRNA and analyze it via microarray.

In other embodiments, measuring the gene expression levels is carried out by detecting protein encoded by each of the genes with antibodies specific to the proteins or by a proteins chip. A protein chip or a protein microarray can assume a variety of formats, but commonly consists of a solid surface onto which enzymes, receptor proteins, antibodies or small molecules are immobilized and used as probes to detect proteins contained in the target sample. In another embodiment, measuring the gene expression levels is carried out by HPLC. Gene expression can also be detected by measuring a characteristic of the gene that affects transcriptional activity of the gene, such as DNA amplification, methylation, mutation and allelic variation. Such methods are known to those skilled in the art.

Other aspects of the invention are kits for conducting the assays described above. Since kits are based on the selection of a set of genes comprising the ones described above, kits are simpler and cheaper than others based on a large amount of genes, such as many commercial microarrays with thousands of probes. Thus, an aspect of the invention refers to the use of a kit for performing the method as defined above, comprising (i) means for measuring the gene expression levels of the selected genes; and (ii) instructions for correlating the gene expression levels above or below pre-determined cut-off levels indicative of the tolerant state in liver transplantation. In a particular embodiment of the invention, the means comprise a microarray or a gene chip which comprises nucleic acid probes, said nucleic acid probes comprising sequences that specifically hybridize to the transcripts of the set of genes defined above. Additionally, the kit further comprises reagents for performing the microarray analysis. In another embodiment, the means comprise oligonucleotide primers for performing a quantitative reverse transcription PCR, said primers comprising sequences that specifically hybridize to the complementary DNA derived from the transcripts of the set of genes defined above. Each such kit would preferably include instructions as well as the reagents typical for the type of assay described. These can include, for example, nucleic acid arrays (e.g. cDNA or oligonucleotide microarrays), as described above, configured to discern the gene expression profile of the invention. They can also contain reagents used to conduct nucleic acid amplification and detection including, for example, reverse transcriptase, reverse transcriptase primer, a corresponding PCR primer set, a thermostable DNA polymerase, such as Taq polymerase, and a suitable detection reagent(s), such as, among others, fluorescent probes or dyes that bind to double-strand DNA such as ethidium bromide or SYBRgreen. Antibody based kits will contain buffers, secondary antibodies, detection enzymes and substrate, e.g. Horse Radish Peroxidase or biotin-avidin based reagents.

Another aspect of the invention refers to the use of a microarray or a gene chip for performing the method as defined above, comprising a solid support and displayed thereon nucleic acid probes which comprises sequences that specifically hybridize to the transcripts of the set of genes defined above.

The practice of the present invention may also employ conventional biology methods and software. Computer software products of the invention typically include computer readable medium having computer-executable instructions for performing the logic steps of the method of the invention. The present invention may also make use of various computer program products and software for a variety of purposes, such as probe design, management of data, analysis, and instrument operation.

Finally, another aspect of the invention refers to a method for selecting or modifying treatment protocol, either before or after liver transplantation is performed, comprising the use of the method of assessing diagnosis and/or prognosis as defined above. Before liver transplantation, the invention permits to identify those patients that will eventually develop tolerance and therefore, can benefit from less aggressive immunosuppression strategies. If liver transplantation has already been done, the invention permits to adequate therapy to the patient status. Patient's therapy can be altered as with additional therapeutics, with changes to the dosage or to the frequency, or with elimination of the current treatment. Such analysis permits intervention and therapy adjustment prior to detectable clinical indicia or in the face of otherwise ambiguous clinical indicia.

As used herein, “the biological sample from the patient” can be whole blood, blood cells (leukocytes), bile fluid or cells there from, and can also include portions of hepatic tissue (in the form of fresh tissue, frozen sections or formalin fixed sections). As is apparent to one of ordinary skilled in the art, samples may be prepared by any available method or process depending on the subsequent analysis. Methods of isolating total mRNA are also well known. Such samples include RNA samples, but also include cDNA synthesized from a mRNA sample isolated from a cell or tissue of interest. Such samples also include DNA amplified from the cDNA, and an RNA transcribed from the amplified DNA.

Throughout the description and claims the word “comprise” and its variations are not intended to exclude other technical features, additives, components, or steps. Additional objects, advantages and features of the invention will become apparent to those skilled in the art upon examination of the description or may be learned by practice of the invention. The following examples are provided by way of illustration, and they are not intended to be limiting of the present invention.

### DETAILED DESCRIPTION OF PARTICULAR EMBODIMENTS

Inventors employed oligonucleotide microarray technology on blood samples from tolerant and non-tolerant liver transplant recipients, and they determined that tolerant liver transplant recipients exhibit a characteristic gene expression profile in peripheral blood. The inventors have finally identified a set of genes through several selection steps whose expression levels can be employed to diagnose the tolerant phenotype with high accuracy.

In order to select the set of genes shown in Table 1, the following methodology was followed by the inventors:

### Patients

Peripheral blood samples were collected from a cohort of 9 tolerant (TOL) recipients of adult cadaveric liver transplants (>1 year of successful immunosuppression discontinuation). For comparison, blood samples were obtained from 11 liver recipients in whom drug weaning was attempted but led to acute rejection requiring reintroduction of immunosuppression (immunosuppression-dependent or non-tolerant, Non-TOL). In NON-TOL recipients blood was harvested at least 2 years after complete resolution of the acute rejection episode. Samples were also collected from 10 age-matched healthy controls.

### Microarray Experiments

After blood mononuclear cell isolation employing a Ficoll-Hypaque layer (Amersham Biosciences, Uppsala, Sweden), total RNA was extracted with Trizol reagent (Life Technologies, Rockville, Md., USA), and the derived cRNA samples were hybridized to Affymetrix Human Genome U133 Plus 2.0 Array containing probes for 47000 transcripts (Affymetrix, Inc, Santa Clara, Calif., USA). The complete database comprised expression measurements of 54675 genes for 9 TOL and 11 ID samples. CEL files were processed, normalized and their expression values summarized using the gcrma algorithm included in bioconductor package (http://www.bioconductor.org). To identify genes differentially expressed between TOL and NON-TOL recipients, data from a subgroup of samples (9 TOL and 8 NON-TOL samples) were first analyzed employing a Bayesian methodology for differential analysis implemented in the program BADGE (Bayesian Analysis of Differential Gene Expression, http://www.genomethods.org/badge). The complete set of data were then independently re-analyzed employing the Significance Analysis of Microarray (SAM; V. G. Tusher et al., Proc. Natl. Acad. Sci. USA. 2001 vol. 98, pp. 5116-21) for 500 permutations. SAM is a method and software for detecting differentially expressed genes in microarray. Its permutation method maintains the inter-gene correlation structure and accounts for the multiple comparison problem automatically. The parameters to accomplish a predicted 5% percentage of False-positives (FDR) were set. Finally, the Prediction Analysis of Microarray program (PAM; R. Tibshirani et al., Proc. Natl. Acad. Sci. USA. 2002, vol. 99, pp. 6567-72) was used to identify the minimum gene set that differentiated TOL from NON-TOL samples. PAM is highly efficient at finding a relatively small number of predictive genes. We apply internal cross-validation and a threshold of 2.9 is used to obtain a selected list with overall error rate 5%.

### Real-Time TaqMan PCR and Flow Cytometry Validation Experiments

To technically validate the microarray results, the expression levels of a set of genes among the 628 genes identified with the program BADGE (CD94, IL1, IL23, TNFα, ICAM1, BAX, BCL-2, CD103, FASL, FOXP3, GITR, GZMB, TIM1, TIM3, HO1, IFNγ, IL10, IL15, TGFβ1, A20, PRF1, IL6) were quantified employing real-time PCR, with an ABI 7900 Sequence Detector System and Assays-on-Demand primer/probe sets (PE Applied Biosystems, Foster City, Calif., USA). For these studies total RNA was treated with DNAse reagent (Ambion, Austin, Tex., USA), and reverse transcription performed using Multiscribed Reverse Transcriptase Enzyme (PE Applied Biosystems). To quantify the levels of mRNA, expression of the target genes were normalized to the housekeeping gene 18S, and data were expressed as relative fold difference between cDNA of the study samples and a calibrated sample. In addition, immunophenotyping studies were performed employing the following antibodies: CD3, CD4, CD8, CD19, CD11c, CD14, CD20, CD28, CD45RA, CD62L, CD123, HLA-DR, αβ-TCR, γδ-TCR, Vδ1-TCR (from BD Biosciences, Mountain View, Calif., USA), Vδ2-TCR, Vα24-TCR (from Beckman Coulter, Fullerton, Calif., USA) and Foxp3 (Treg staining kit, eBioscience, San Diego, Calif., USA). For staining, aliquots of 100 μl of EDTA-anticoagulated blood were incubated at room temperature for 15 min with a combination of the appropriate antibodies. Stained samples were analyzed using a FACScalibur flow cytometer with Cellquest Pro software (BD Biosciences).

### Global Gene Expression Profiling can Discriminate Between TOL and Non-TOL Recipients

In the first analysis utilizing a subgroup of samples, BADGE analysis selected the genes with more than 99.5% and less than 0.5% chances of being more expressed in TOL as compared to NON-TOL recipients. This resulted in a total of 462 positively and 166 negatively changed genes. Among these 628 genes, expression levels of a set of genes (CD94, IL1, IL23, TNFα, ICAM1, BAX, BCL-2, CD103, FASL, FOXP3, GITR, GZMB, TIM1, TIM3, HO1, IFNγ, IL10, IL15, TGFβ1, A20, PRF1, IL6) were quantified by real-time PCR in order to technically validate the microarray accuracy. In all cases trends were similar to the gene expression patterns identified by the microarrays. These confirmatory PCR experiments indicate the feasibility of employing real-time PCR to directly assess the expression levels of the selected list of differentially expressed genes, without the need to perform whole-genome microarray studies.

In order to evaluate the statistical significance of the differentially expressed genes in the complete data set, we then employed SAM setting the parameters to accomplish a predicted 5% percentage FDR. This yielded a list of 68 statistically significant genes that could discriminate between TOL and NON-TOL samples with an accuracy of 95%. Independently, PAM was used on the same complete data set to find the smallest set of genes capable of accurately classifying samples in the TOL and NON-TOL categories. This resulted in a list of 34 probes fitting the 5% overall error rate criteria. Of our 68 probes from the SAM gene-set, 22 were also found by the PAM method. Thus, 44 of the probes from SAM approach are not required for classification. Additional computational cross-validation established that the consensuated list of 22 genes could classify samples within the TOL and NON-TOL categories with a 5% error rate. The following table (TABLE 1) shows the list of 22 genes that constitutes the minimal gene-set capable of discriminating between TOL and NON-TOL samples.

Flow cytometry experiments further validated the microarray results, in which only gamma delta T lymphocytes (expressing TRD@) among peripheral blood cells, were found to discriminate between TOL and NON-TOL recipients.

TABLE 1

| | Probe set ID | | Gene |
|---|---|---|---|
| Gene ID | (Affymetrix) | Gene name | symbol |
| 7049 | 240188_at | Transforming growth factor, beta receptor III | TGFBR3 |
| | | (betaglycan) | |
| 3820 | 214470_at | killer cell lectin-like receptor subfamily B, | KLRB1 |
| | | member 1 | |
| 79053 | 203545_at | asparagine-linked glycosylation 8 homolog | ALG8 |
| 2189 | 203564_at | Fanconi anemia, complementation group G | FANCG |
| 79760 | 1555751_a_at | gem (nuclear organelle) associated protein 7 | GEMIN7 |
| 4818 | 213915_at | natural killer cell group 7 sequence | NKG7 |
| 5887 | 214422_at | RAD23 homolog B (S. cerevisiae) | RAD23B |
| 57823 | 219159_s_at | SLAM family member 7 | SLAMF7 |
| 51499 | 218403_at | TP53 regulated inhibitor of apoptosis 1 | TRIAP1 |
| 5495 | 209296_at | protein phosphatase 1B (formerly 2C), | PPM1B |
| | | magnesium-dependent, beta isoform | |
| 79892 | 217905_at | chromosome 10 open reading frame 119 | C10orf119 |
| 6964 | 217143_s_at | T cell receptor delta locus | TRD@ |
| 54433 | 219110_at | nucleolar protein family A, member 1 (H/ACA | NOLA1 |
| | | small nucleolar RNPs) | |
| 54165 | 222679_s_at | DCN1, defective in cullin neddylation 1, domain | DCUN1D1 |
| | | containing 1 (S. cerevisiae) | |
| 84062 | 223763_at | dystrobrevin binding protein 1 | DTNBP1 |
| 79158 | 212959_s_at | N-acetylglucosamine-1-phosphate transferase, | GNPTAB |
| | | alpha and beta subunits | |
| 10213 | 212296_at | proteasome (prosome, macropain) 26S subunit, | PSMD14 |
| | | non-ATPase, 14 | |
| 22818 | 222386_s_at | coatomer protein complex, subunit zeta 1 | COPZ1 |
| 6281 | 238909_at | S100 calcium binding protein A10 (annexin II | S100A10 |
| | | ligand, calpactin I, light polypeptide) | |
| 25814 | 208833_s_at | ataxin 10 | ATXN10 |
| 2926 | 201520_s_at | G-rich RNA sequence binding factor 1 | GRSF1 |
| 51744 | 220307_at | CD244 molecule, natural killer cell receptor 2B4 | CD244 |

In order to identify the additional genes shown in Tables 2 and 3 the following methodology was followed by the inventors:

### Patients

Peripheral blood samples were collected from a cohort of 17 tolerant (TOL) recipients of adult cadaveric liver transplants (>1 year of successful immunosuppression discontinuation), and 21 Non-TOL liver recipients (recipients in whom drug weaning was attempted but led to acute rejection requiring reintroduction of immunosuppression, with blood being harvested at least 1 year after complete resolution of the acute rejection episode). In addition, blood samples were also collected from 16 age-matched healthy individuals and from 15 stable liver transplant recipients under maintenance immunosuppression (STA) in whom blood collection was performed before immunosuppression treatment was withdrawn (10 of the STA patients eventually rejected and turned out to be Non-TOL, while 5 did not reject and were therefore considered as TOL).

### Microarray Experiments and Data Analysis

Microarray data from samples of transplanted patients (17 TOL, 21 Non-TOL and 15 STA) were normalised using the GC content adjusted-robust multi-array (GC-RMA) algorithm, which computes expression values from probe intensity values incorporating probe sequence information (cf. Z. Wu, et al., “A Model Based Background Adjustment for Oligonucleotide Expression Arrays”, J.A.S.A. 2004, vol. 99, pp. 909-17). Next a conservative probe-filtering step eliminating those probes with a maximum expression value lower than 5 was employed, which resulted in a number of probes out of the original probe set. In order to eliminate non-biological experimental variation or batch effects observed across successive batches of microarray experiments the ComBat approach was applied, which uses nonparametric empirical Bayes frameworks to adjust data more correctly (cf. W. E. Johnson, et al., “Adjusting batch effects in microarray expression data using empirical Bayes methods”, Biostatistics 2007, vol 8, pp. 118-27). Significant Analysis of Microarray (SAM) was used to identify genes differentially expressed between the TOL and Non-TOL groups (17 and 21 samples respectively). SAM uses modified t test statistics for each gene of a dataset and a fudge factor to compute the t value, thereby controlling for unrealistically low standard deviations for each gene. Furthermore SAM allows control of the false discovery rate (FDR) by selecting a threshold for the difference between the actual test result and the result obtained from repeated permutations of the tested groups. Differential gene expresión was further explored by using the nearest shrunken centroid classifier implemented in the Predictive Analysis of Microarray (PAM) package to identify the minimal set of genes capable of predicting the tolerant state with an error rate <5%. This method incorporates an internal cross-validation step during feature selection in which the model is fit on 90% of the samples and then the class of the remaining 10% is predicted. This procedure is repeated 10 times to compute the overall error (ten-fold cross-validation). The PAM classifier was then used on the 38-sample set to perform multidimensional scaling analysis, and employed to predict class in the set of 15 samples obtained from STA patients. Additional classification models were constructed employing the misclassification penalized posterior (MiPP) algorithm (cf. M. Soukup et al., “Robust classification modeling on microarray data using misclassification penalized posterior”, Bioinformatics 2005, vol 21 p.p: i423-30). MiPP is a recently developed method for assessing the performance of a prediction model that computes the sum of the posterior classification probabilities penalized by the number of incorrectly classified samples. In this study MiPP was applied to the subset of genes selected by SAM at 5% FDR. The sequential selection of genes was made using the linear discriminant analysis (LDA) and/or linear-super vector machine learning classifiers (linear-SVM) together with a 10-fold cross-validation algorithm. MiPP implements an additional random-split internal validation strategy that is particularly well suited to datasets such as this one in which there is no clear distinction between training and independent test sets. In this random sampling procedure the full dataset is randomly split multiple times into training (⅔ of samples) and test (⅓ of samples) sets. This is repeated 100 times, and for each split a parsimonious gene model is identified and further evaluated by 100 additional independent splits.

### Differential Gene Expression Between TOL and Non-TOL Samples Employing Microarrays

Comparison of TOL and Non-TOL microarray data employing SAM yielded a total of 2341 and 88 probes with a FDR <5% and <%1, respectively. PAM analysis performed in parallel on the same two groups of patients resulted in the identification of a subset of 25 probes (corresponding to 23 genes all of them present in the SAM list) capable of correctly classifying the 17 tolerant and 21 non-tolerant recipients with an overall error rate of 0.026 (sensibility 1, specificity 0.914). Independent validation of the predictive capacity of this gene set was performed classifying the 15 STA patients employing PAM, Supervector Machine Learning (SVA) and Liner Discriminant Analysis (LDA) prediction algorithms. All 15 STA patients were correctly classified regardless of the class prediction tool employed. In order to determine if a prediction model including a minimum set of features could be obtained, we then re-analyzed the probes identified by SAM at 5% FDR utilizing a MiPP-based classification modelling approach. MiPP identified 90 classifiers comprising 2-10 genes exhibiting overall error rates <10%. Seventeen out of the 23 genes identified by PAM were present among the list of most parsimonious genes as assessed by MiPP. Overall, data analysis performed employing three different algorithms results in a series of concordant predictive models that contain a very limited number of features and exhibit high accuracy at discriminating TOL from Non-TOL samples.

### Real-Time TaqMan PCR Experiments

The expression pattern of a group of 56 target genes and 4 housekeeping genes (18S, GUS, HPRT and GAPDH) was quantified employing the ABI 7900 Sequence Detector System and LDA PCR cards (PE Applied Biosystems, Foster City, Calif., USA) on peripheral blood samples obtained from 16 Non-TOL, 16 TOL (selected among the 17 TOL and 21 Non-TOL patients in whom microarray experiments had been performed) and 16 healthy individuals individuals. For all PCR experiments total RNA was treated with DNAse reagent (Ambion, Austin, Tex., USA), and reverse transcription performed using Multiscribed Reverse Transcriptase Enzyme (PE Applied Biosystems). Target genes were selected based on their high differential expression between TOL and Non-TOL samples in the microarray experiments employing SAM, PAM and MiPP as previously described. To quantify the levels of mRNA we normalized expression of the target genes to the housekeeping gene HPRT1 and expressed the results as relative expression between cDNA of the target samples and a calibrated sample according to the ΔΔC_{T} method (results were similar when normalization was carried out employing the remaining 3 housekeeping genes). Results were analyzed employing an ANOVA test. In addition, both PAM and MiPP algorithms were utilized to select the minimal set of target genes with the lowest possible classification error. PAM analysis of the PCR data set resulted in the identification of 7 genes which are shown in Table 3. MiPP analysis identified 23 genes (Table 2), different from the original 22 genes shown in Table 1, which also exhibited very high predictive capacity.

In summary, in order to select the additional genes shown in Tables 2 and 3, the inventors have increased the number of patients (tolerant and non-tolerant) used for gene screening by microarray, have employed a new statistical method for gene selection, and have validated all results coming out from the microarray analysis employing quantitative PCR techniques.

TABLE 2

| | | NCBI |
|---|---|---|
| Gene | | Gene |
| Symbol | Gene Name | ID |
| CTBP2 | C-terminal binding protein 2 | 1488 |
| CLIC3 | chloride intracellular channel 3 | 9022 |
| KLRF1 | killer cell lectin-like receptor subfamily F, | 51348 |
| | member 1 | |
| IL2RB | interleukin 2 receptor, beta | 3560 |
| OSBPL5 | oxysterol binding protein-like 5 | 114879 |
| FEZ1 | fasciculation and elongation protein zeta 1 (zygin I) | 9638 |
| FLJ14213 | hypothetical protein FLJ14213 | 79899 |
| CD160 | CD160 molecule | 11126 |
| RGS3 | regulator of G-protein signaling 3 | 5998 |
| CX3CR1 | chemokine (C-X3-C motif) receptor 1 | 1524 |
| PTGDR | prostaglandin D2 receptor (DP) | 5729 |
| CD9 | CD9 molecule | 928 |
| PDE4B | phosphodiesterase 4B, cAMP-specific | 5142 |
| | (phosphodiesterase E4 dunce homolog, Drosophila) | |
| ERBB2 | v-erb-b2 erythroblastic leukemia viral oncogene | 2064 |
| | homolog 2, neuro/glioblastoma derived oncogene | |
| | homolog (avian) | |
| FEM1C | fem-1 homolog c (C. elegans) | 56929 |
| WDR67 | WD repeat domain 67 | 93594 |
| ZNF267 | zinc finger protein 267 | 10308 |
| ZNF295 | zinc finger protein 295 | 49854 |
| EPS8 | epidermal growth factor receptor pathway | 2059 |
| | substrate 8 | |
| IL8 | interleukin 8 | 3576 |
| NCALD | neurocalcin delta | 83988 |
| NOTCH2 | Notch homolog 2 (Drosophila) | 4853 |
| PTCH1 | patched homolog 1 (Drosophila) | 5727 |

TABLE 3

| Gene | | |
|---|---|---|
| Symbol | Gene Name | NCBI Gene ID |
| CTBP2 | C-terminal binding protein 2 | 1488 |
| CLIC3 | chloride intracellular channel 3 | 9022 |
| KLRF1 | killer cell lectin-like receptor subfamily F, | 51348 |
| | member 1 | |
| IL2RB | interleukin 2 receptor, beta | 3560 |
| OSBPL5 | oxysterol binding protein-like 5 | 114879 |
| FEZ1 | fasciculation and elongation protein zeta 1 | 9638 |
| | (zygin I) | |
| SLAMF7 | SLAM family member 7 | 57823 |
| CD160 | CD160 molecule | 11126 |

The selection of the most relevant genes shown in Table 3 and whose expression should be preferably measured, was based on sensitivity and specificity parameters. Table 4 shows the individual values for each of the aforesaid seven genes included in Table 3.

TABLE 4

| Gene symbol | Sensitivity | Specificity | Mean Error Rate |
|---|---|---|---|
| FEZ1 | 0.81 | 1.00 | 0.91 |
| CTBP2 | 0.75 | 1.00 | 0.88 |
| CLIC3 | 0.81 | 0.93 | 0.87 |
| SLAMF7 | 0.81 | 0.93 | 0.87 |
| KLRF1 | 0.88 | 0.80 | 0.84 |
| OSBPL5 | 0.75 | 0.87 | 0.81 |
| IL2RB | 0.94 | 0.67 | 0.80 |
| CD160 | 0.81 | 0.80 | 0.81 |

Sensibility (Sens) and Specificity (Spec) and Mean Error Rate (MER) values were calculated as follows:

Sens=TP/TP+FN

Spec=TN/TN+FP

Mean Error Rate=Σ(FN+FP)/(TP+TN+FP+FN)/N

### Where:

TP=true-positive;
TN=true-negative;
FN=false-negative;
FP=false-positive.
N=number of samples measured

As a way of example, a value of 0.81 of sensitivity means that 81% of the tolerant patients has a given gene above or below the predetermined cut-off levels. A value of 1.00 of specificity means that none of non-tolerant patients has the expression of the given gene above or below the predetermined cut-off levels. Concerning MER, a value of 0.054 means that only 5.4% of the samples wherein the expression of a give gene is above or below the predetermined cut-off levels. are not correctly identified.

Also, expression of combinations of any of the genes of said subset among themselves or with other genes pertaining either to Table 1 or 2, gives mean error rates which qualify those combinations as highly relevant for the assessment of diagnosis/prognosis of tolerance to the transplant. In Table 5, combinations of genes resulting in mean error rates ≦8% may be shown.

TABLE 5

| Gene Symbols | | | | | | | MER |
|---|---|---|---|---|---|---|---|
| CTBP2 | CLIC3 | FANCG | CD160 | C10orf119 | WDR67 | | 0.054 |
| CTBP2 | IL2RB | GEMIN7 | GNPTAB | RGS3 | CLIC3 | SLAMF7 | 0.055 |
| CLIC3 | CTBP2 | WDR67 | PDE4B | PTGDR | | | 0.058 |
| CLIC3 | CD244 | PDE4B | WDR67 | NOTCH2 | PTGDR | | 0.062 |
| CTBP2 | CLIC3 | GEMIN7 | FEZ1 | PDE4B | WDR67 | | 0.063 |
| CTBP2 | CLIC3 | WDR67 | C10orf119 | PDE4B | | | 0.063 |
| CD160 | FLJ14213 | | | | | | 0.064 |
| EPS8 | IL2RB | | | | | | 0.064 |
| CLIC3 | PDE4B | WDR67 | | | | | 0.065 |
| FEZ1 | CLIC3 | CX3CR1 | EPS8 | CTBP2 | SLAMF7 | | 0.065 |
| CLIC3 | WDR67 | CTBP2 | CD160 | ZNF295 | SLAMF7 | | 0.066 |
| CLIC3 | OSBPL5 | EPS8 | PTCH1 | SLAMF7 | FEZ1 | | 0.066 |
| FEZ1 | CLIC3 | PDE4B | WDR67 | | | | 0.069 |
| CLIC3 | EPS8 | WDR67 | PDE4B | | | | 0.07 |
| CLIC3 | CTBP2 | SLAMF7 | IL2RB | | | | 0.071 |
| SLAMF7 | KLRB1 | C10orf119 | COPZ1 | GRSF1 | | | 0.073 |
| SLAMF7 | KLRF1 | RGS3 | CTBP2 | | | | 0.074 |
| CTBP2 | IL2RB | FEZ1 | CLIC3 | | | | 0.074 |
| CTBP2 | CD160 | CX3CR1 | CLIC3 | | | | 0.075 |
| FEZ1 | CLIC3 | CTBP2 | | | | | 0.075 |
| CLIC3 | PDE4B | WDR67 | | | | | 0.075 |
| CLIC3 | FEZ1 | CTBP2 | SLAMF7 | | | | 0.077 |
| CTBP2 | CLIC3 | | | | | | 0.078 |
| CLIC3 | CTBP2 | | | | | | 0.078 |
| FEZ1 | CTBP2 | CLIC3 | KLRF1 | IL2RB | | | 0.078 |
| CLIC3 | CX3CR1 | WDR67 | | | | | 0.078 |
| CLIC3 | CTBP2 | | | | | | 0.079 |
| FEZ1 | CLIC3 | | | | | | 0.079 |
| CTBP2 | CLIC3 | | | | | | 0.08 |
| CLIC3 | KLRF1 | SLAMF7 | | | | | 0.08 |
| CTBP2 | CLIC3 | | | | | | 0.08 |

1. A method of assessing diagnosis and/or prognosis of the tolerant state in liver transplantation in a human patient, comprising the steps of:
(a) obtaining a biological sample from the patient; and
(b) measuring the expression levels in the sample of a set of genes comprising the following twenty two:
transforming growth factor beta receptor III (TGFBR3, NCBI Gene ID 7049), killer cell lectin-like receptor subfamily B member 1 (KLRB1, NCBI Gene ID 3820), asparagine-linked glycosylation 8 homolog (ALG8, NCBI Gene ID 79053), Fanconi anemia complementation group G (FANCG, NCBI Gene ID 2189), gem associated protein 7 (GEMIN7, NCBI Gene ID 79760), natural killer cell group 7 sequence (NKG7, NCBI Gene ID 4818), RAD23 homolog B of Saccharomyces cerevisiae (RAD23B, NCBI Gene ID 5887), SLAM family member 7 (SLAMF7, NCBI Gene ID 57823), TP53 regulated inhibitor of apoptosis 1 (TRIAP1, NCBI Gene ID 51499), protein phosphatase 1B magnesium-dependent beta isoform (PPM1B, NCBI Gene ID 5495), chromosome 10 open reading frame 119 (C10orf119, NCBI Gene ID 79892), T cell receptor delta locus (TRD@, NCBI Gene ID 6964), nucleolar protein family A member 1 (NOLA1, NCBI Gene ID 54433), DCN1 defective in cullin neddylation 1 domain containing 1 of Saccharomyces cerevisiae (DCUN1D1, NCBI Gene ID 54165), dystrobrevin binding protein 1 (DTNBP1, NCBI Gene ID 84062), N-acetylglucosamine-1-phosphate transferase alpha and beta subunits (GNPTAB, NCBI Gene ID 79158), proteasome 26S subunit non-ATPase 14 (PSMD14, NCBI Gene ID 10213), coatomer protein complex subunit zeta 1 (COPZ1, NCBI Gene ID 22818), S100 calcium binding protein A10 (S100A10, NCBI Gene ID 6281), ataxin 10 (ATXN10, NCBI Gene ID 25814), G-rich RNA sequence binding factor 1 (GRSF1, NCBI Gene ID 2926), and CD244 molecule natural killer cell receptor 2B4 (CD244, NCBI Gene ID 51744); wherein the corresponding gene expression levels above or below predetermined cut-off levels are indicative of the tolerant state in liver transplantation.

2. The method according to claim 1, wherein the gene expression levels are above pre-determined cut-off levels obtained from a control sample.

3. The method according to claim 2, wherein the control sample is obtained from a non-tolerant liver transplant recipient requiring on-going immunosuppression therapy.

4. The method according to any of the claims 1-3, wherein measuring the gene expression levels is carried out using a microarray or a gene chip which comprises nucleic acid probes, said nucleic acid probes comprising sequences that specifically hybridize to the transcripts of the corresponding set of genes.

5. The method according to claim 4, wherein the microarray is a cDNA microarray.

6. The method according to claim 4, wherein the microarray is an oligonucleotide microarray.

7. The method according to any of the claims 1-3, wherein measuring the gene expression levels is carried out by quantitative reverse transcription polymerase chain reaction of RNA extracted from the sample.

8. The method according to any of the claims 1-3, wherein measuring the gene expression levels is carried out by detecting the proteins encoded by the corresponding genes.

9. The method according to claim 8, wherein the proteins are detected by antibodies specific to the proteins.

10. The method according to claim 8, wherein the proteins are detected by a proteins chip.

11. The method according to any of the claims 1-3, wherein measuring the gene expression levels is carried out by HPLC.

12. Use of a kit for performing the method as defined in claims 1-11, comprising (i) means for measuring the gene expression levels of the corresponding set of genes; and (ii) instructions for correlating the gene expression levels above or below pre-determined cut-off levels indicative of the tolerant state in liver transplantation.

13. Use of the kit according to claim 12, wherein the means comprise a microarray or a gene chip which comprises nucleic acid probes, said nucleic acid probes comprising sequences that specifically hybridize to the transcripts of the corresponding set of genes.

14. Use of the kit according to claim 13, further comprising reagents for performing a microarray analysis.

15. Use of the kit according to claim 12, wherein the means comprise oligonucleotide primers for performing a quantitative reverse transcription polymerase chain reaction, said primers comprising sequences that specifically hybridize to the complementary DNA derived from the transcripts of the corresponding set of genes.

16. Use of a microarray or a gene chip for performing the method as defined in claims 2-5, comprising a solid support and displayed thereon nucleic acid probes which comprises sequences that specifically hybridize to the transcripts of the corresponding set of genes.

17. Method for selecting or modifying treatment protocol, either before or after liver transplantation is performed, comprising the use of the method as defined in claims 1-11.

18. A method of assessing diagnosis and/or prognosis of the tolerant state in liver transplantation in a human patient, according to any of the claims 1-11, wherein (b) measuring the expression levels in the sample of a set of genes, further comprises the following 23:

| | | |
|---|---|---|
| CTBP2 | C-terminal binding protein 2 | 1488 |
| CLIC3 | chloride intracellular channel 3 | 9022 |
| KLRF1 | killer cell lectin-like receptor subfamily F, | 51348 |
| | member 1 | |
| IL2RB | interleukin 2 receptor, beta | 3560 |
| OSBPL5 | oxysterol binding protein-like 5 | 114879 |
| FEZ1 | fasciculation and elongation protein zeta 1 | 9638 |
| | (zygin I) | |
| FLJ14213 | hypothetical protein FLJ14213 | 79899 |
| CD160 | CD160 molecule | 11126 |
| RGS3 | regulator of G-protein signaling 3 | 5998 |
| CX3CR1 | chemokine (C-X3-C motif) receptor 1 | 1524 |
| PTGDR | prostaglandin D2 receptor (DP) | 5729 |
| CD9 | CD9 molecule | 928 |
| PDE4B | phosphodiesterase 4B, cAMP-specific | 5142 |
| | (phosphodiesterase E4 dunce homolog, | |
| | Drosophila) | |
| ERBB2 | v-erb-b2 erythroblastic leukemia viral | 2064 |
| | oncogene homolog 2, neuro/glioblastoma | |
| | derived oncogene homolog (avian) | |
| FEM1C | fem-1 homolog c (C. elegans) | 56929 |
| WDR67 | WD repeat domain 67 | 93594 |
| ZNF267 | zinc finger protein 267 | 10308 |
| ZNF295 | zinc finger protein 295 | 49854 |
| EPS8 | epidermal growth factor receptor pathway | 2059 |
| | substrate 8 | |
| IL8 | interleukin 8 | 3576 |
| NCALD | neurocalcin delta | 83988 |
| NOTCH2 | Notch homolog 2 (Drosophila) | 4853 |
| RGS3 | Regulator of G-protein Signalling 3 | 5998 |
| PTCH1 | patched homolog 1 (Drosophila) | 5727 |

wherein the corresponding gene expression levels above or below pre-determined cut-off levels are indicative of the tolerant state in liver transplantation.

19. A method of assessing diagnosis and/or prognosis of the tolerant state in liver transplantation in a human patient, according to any of the claim 1-11 or 18, wherein (b) measuring the expression levels in the sample of a set of genes, at least comprises gene SLAMF7.

20. A method of assessing diagnosis and/or prognosis of the tolerant state in liver transplantation in a human patient, wherein (b) measuring the expression levels in the sample of a set of genes, comprises the following eight genes or combinations thereof:

| | | |
|---|---|---|
| CTBP2 | C-terminal binding protein 2 | 1488 |
| CLIC3 | chloride intracellular channel 3 | 9022 |
| KLRF1 | killer cell lectin-like receptor subfamily F, | 51348 |
| | member 1 | |
| IL2RB | interleukin 2 receptor, beta | 3560 |
| OSBPL5 | oxysterol binding protein-like 5 | 114879 |
| FEZ1 | fasciculation and elongation protein zeta 1 | 9638 |
| | (zygin I) | |
| SLAMF7 | SLAM family member 7 | 57823 |
| CD160 | CD160 molecule | 11126 |

wherein the corresponding gene expression levels above or below pre-determined cut-off levels are indicative of the tolerant state in liver transplantation.

21. A method of assessing diagnosis and/or prognosis of the tolerant state in liver transplantation in a human patient, wherein (b) measuring the expression levels in the sample of a set of genes, comprises at least one gene from the set of eight genes of claim 20 in combination with one or more genes selected from the 22 genes of claim 1 and/or from the 23 genes of claim 18

22. Use of a kit for performing the method as defined in claims 18-21, comprising (i) means for measuring the gene expression levels of the corresponding set of genes; and (ii) instructions for correlating the gene expression levels above or below pre-determined cut-off levels indicative of the tolerant state in liver transplantation.

23. Use of the kit according to claim 22, wherein the means comprise a microarray or a gene chip which comprises nucleic acid probes, said nucleic acid probes comprising sequences that specifically hybridize to the transcripts of the corresponding set of genes.

24. Use of the kit according to claim 23, further comprising reagents for performing a microarray analysis.

25. Use of the kit according to claim 22, wherein the means comprise oligonucleotide primers for performing a quantitative reverse transcription polymerase chain reaction, said primers comprising sequences that specifically hybridize to the complementary DNA derived from the transcripts of the corresponding set of genes.

26. Use of a microarray or a gene chip for performing the method as defined in claims 18-21, comprising a solid support and displayed thereon nucleic acid probes which comprises sequences that specifically hybridize to the transcripts of the corresponding set of genes.

27. Method for selecting or modifying treatment protocol, either before or after liver transplantation is performed, comprising the use of the method as defined in claims 18-21.
